(19) United States
(12) Patent Application Publication
(10) Pub. No.: US 2008/0275307 A1
(43) Pub. Date: Nov. 6, 2008

(54) Telemetry System With out of Range Notification Features

(75) Inventor: Susanne M. Poschmann, Andover, MA (US)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 10/597,567

(22) PCT Filed: Jan. 28, 2005
(86) PCT No.: PCT/IB2005/050384

§ 371 Date: Jul. 31, 2006

Related U.S. Application Data
(60) Provisional application No. 60/542,404, filed on Feb. 6, 2004.

(51) Int. Cl. A61B 5/00

(52) U.S. Cl. 600/300; 340/573.1

(57) ABSTRACT

An apparatus and method for notifying users of a telemetry device as they move out of the coverage area are provided. As the user of the telemetry device has moved out of the coverage area, the device notifies the user after attempting to establish a connection with the network for a predetermined time period, by transmitting an audio signal and/or displaying a visual signal.

The present invention provides a system and method for alerting a patient when he or she moves out of the monitoring range of a medical telemetry system.

Medical telemetry systems that enable physiological data of remotely located patients to be monitored from a central location are well known in the art. Unless these ambulatory patients are continuously supervised, they may wander out of the system's coverage area leaving them ummonitored. Although alerts are generated at a central nursing station, currently there is no means of alerting individuals with the device that they have left the coverage area.

Therefore, there is a need in the art for alerting patients as they move out of a coverage area.

The present invention overcomes the above-described problem, and provides additional advantages, by providing a method and apparatus for notifying the users of a telemetry device as they move out of a coverage area of a network. In particular, the user is notified after losing a connection with the network or after a specified period has expired in attempting to reestablish a connection with the network, by receiving an audio and/or visual signal from the telemetry device. Telemetry may be equipped with means for establishing a connection with a central monitoring station, means for processing patient data collected by a respective telemetry device, and means for transmitting an audio and/or visual signal as the user moves out of the coverage area of the network.

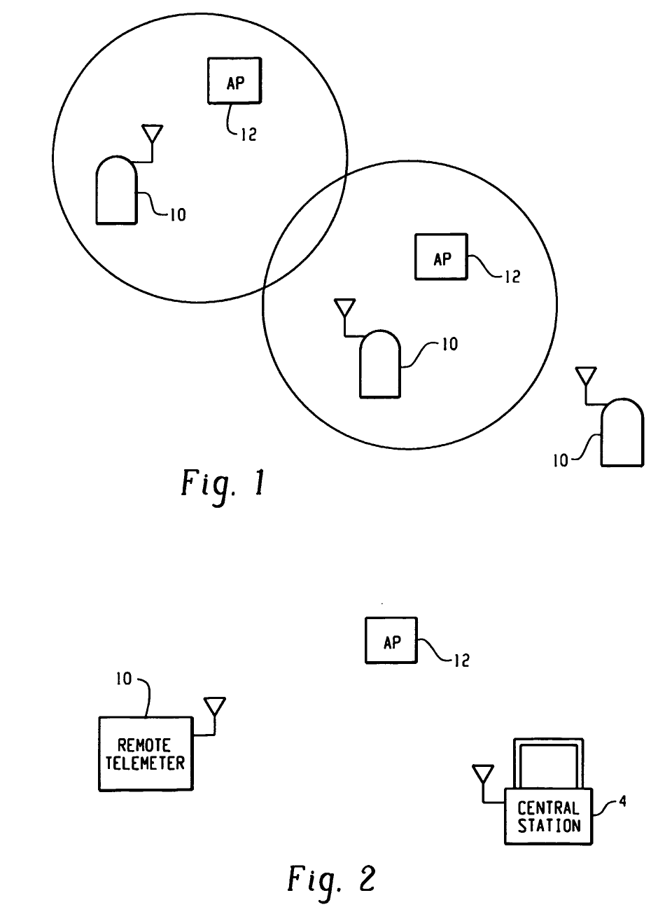

FIG. 1 is a simplified block diagram illustrating a networking environment whereto embodiments of the present invention are to be applied;

FIG. 2 illustrates another view depicting the system whereto embodiments of the present invention are to be applied; and,

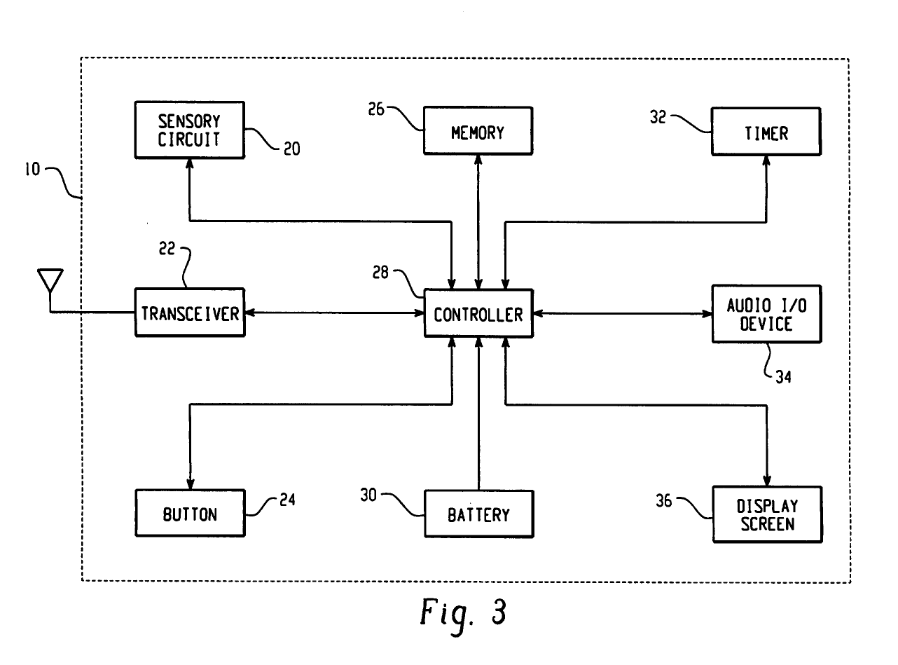

FIG. 3 is a simplified block diagram illustrating the telemetry device in accordance with the present invention.

It is to be understood by persons of ordinary skill in the art that the following descriptions are provided for purposes of illustration and not for limitation. An artisan understands that there are many variations that lie within the spirit of the invention and the scope of the appended claims. Unnecessary detail of known functions and operations may be omitted from the current description so as not to obscure the present invention.

FIG. 1 illustrates a representative network whereto embodiments of the present invention are to be applied. As shown in FIG. 1, communication typically takes place between the telemetry device 10 and the access point (AP) 12, which is within the same radio coverage area. Each telemetry device 10 senses physiological data of a respective patient and transmits it to a central station via the access point 12. The physiologic data may include numeric data such as blood pressure, CO₂, temperature readings, ECG signals, and other patient-related data.

Referring to FIG. 2, a central monitoring station 14 communicates via radio frequency with a plurality of battery-powered remote telemetry device 10 via an access point 12 for monitoring purposes. The central station 14 displays the patient data it receives and also transmits various control signals to each telemetry device 10. The central station 14 may include a personal computer (PC) and executes medical-monitoring software that is adapted to the needs of the network in which the system is installed. The transmission of data between the central station 14 and each telemetry device 10 may be achieved using a time-divisional multiple access (TDMA) scheme, Code Division Multiple Access (CDMA) scheme, frequency-divisional multiple access (FDMA), or other conventional modulation schemes known to artisans. Further, to provide a greater coverage area, multiple access points 12 and central stations 14 can be strategically provided through the desired coverage area, with the central stations 14 interconnected by a wired local area network (LAN).

Referring to FIG. 3, each telemetry device 10 may include a sensory circuit 20, a transceiver 22, a button 24, a memory 26, a controller 28, a battery 30, a timer 32, an audio module 34, and a display screen 36.

The sensory circuit 20 is coupled to the controller 28 to receive patient data, which may be stored in the memory 26, to process, and to communicate to the central station 14. The controller 28 communicates with the central station via the transceiver 22, which may be any of a variety of wireless transceivers known to those skilled in the art. The transceiver 22 is coupled to an antenna and is configured to communicate with the access point 12 using the appropriate communications protocol and to transmit data received from the controller 28 with the central station 14.

The controller 28 is also coupled to the button 24, which is operable by a patient to input information to the controller 28, as well as to control the controller 28. Each button may have a single or multiple functions depending on such factors as the operating condition of the controller 28.

The controller 28 is operably coupled to receive power from a battery 30 and coupled to a memory 26. The memory 26 may be any one or a combination of devices adapted to store electronic information such as RAM, ROM, PROM, EPROM, etc. The memory 26 may be configured to store one or more software-control programs executable by controller 28 to perform its various functions including receiving and analyzing vital data, presenting information to a user, etc. In addition, the software may include programs necessary for communicating with the central station 14.

The controller 28 is coupled to the timer 32 for counting purposes and coupled to the audio device 34 to transmit audio signals or messages to the user. The controller 28 is further configured to control display screen 36 to display an image representing the patient data. It will be appreciated that the image displayed on the display screen 36 will vary depending on the patient data collected by the telemetry device 10.

In operation, the telemetry device 10 establishes communications with the central station 14 by transmitting a request for communications to the central station 14 via the access point 12. It will be appreciated by those of skill in the art that a telemetry device 10 may broadcast a request for communications in any of a variety of ways. The request signal may include a variety of parameters adapted to notify the central station 14 about the telemetry device 10. For example, a communications request may include the type, identity, and other capabilities of the telemetry device 10. The access point 12, or other network devices and routers, pass the communications request to the central station 14. Upon receiving the communications request, the central station 14 sends an acknowledgement back to the telemetry device 10, thus establishing a connection for a monitoring session.

Meanwhile, the transceiver 22 is coupled to an antenna to convert received patient data detected by the sensory circuit 20 and transmit it to the central station 14 via the access point 12 under the control of the controller 28. As is known to those of skill in the art, communications between the telemetry device 10 and the central station 14 may be lost for a variety of reasons. For example, the patient is moved out of the range of the access point 12. In such event, the telemetry device 10 continues to attempt to establish a connection with the access point 12 by transmitting a request for communications to the central station 14. If a predetermined time period has passed after disconnection with the central station 14, the controller 28 alerts the patient by sending an audio and/or visual signal to inform him/her of the dropped communication via audio device 34 and display screen 36. Here, the timer 32 may be used to time-out an attempt to connect, or, alternatively, a number of connection attempts when the attempt to connect fails, but each type of network that can be connected by the telemetry device will have a connection protocol that determines how and for how long or for how many attempts a connection is attempted prior to notifying the user. In a preferred embodiment, these connection parameters are stored in the memory 26 and can be updated by download or interactively from the user interface.

Having thus described a preferred embodiment of a system and method for alerting the user of a telemetry device when he or she is out of the coverage area, it should be apparent to those skilled in the art that certain advantages of the system have been achieved. The foregoing is to be constructed as only being an illustrative embodiment of this invention. Persons skilled in the art can easily conceive of alternative arrangements providing a function similar to this embodiment without any deviation from the fundamental principles or the scope of this invention.

In addition, many modifications may be made to adapt the teaching of the present invention to a particular situation without departing from its central scope. Therefore it is intended that the present invention not be limited to the particular embodiments disclosed as the best mode contemplated for carrying out the present invention, but that the present invention include all embodiments falling within the scope of the appended claims.

1. A telemetry apparatus for notifying a user of moving out of a coverage area, comprising:
means for establishing a connection with a network in communication with a central monitoring station; and
means for transmitting an audio signal as the user moves out of the coverage area of the network.

2. The telemetry apparatus of claim 1, further comprising means for collecting patient data of the user.

3. The telemetry apparatus of claim 2, further comprising means for processing the patient data for a subsequent transmission to the central monitoring station.

4. The telemetry apparatus of claim 1, further comprising means for displaying a visual signal as the user moves out of the coverage area of the network.

5. The telemetry apparatus of claim 4, wherein the visual signal is transmitted after a predetermined time period has expired.

6. The telemetry apparatus of claim 4, wherein the visual signal is transmitted after a predetermined number of attempts to reconnect to the network has failed.

7. The telemetry apparatus of claim 1, wherein the audio signal is transmitted after a predetermined time period has expired.

8. The telemetry apparatus of claim 1, wherein the audio signal is transmitted after a predetermined number of attempts to reconnect to the network has failed.

9. A system for notifying a user of moving out of a coverage area, comprising:
a central monitoring station wirelessly communicating with a plurality of telemetry devices for processing patient data;
wherein, said telemetry device includes:
means for establishing a connection with the central monitoring station; and
means for transmitting an audio or visual signal as the user moves out of the coverage area of the network.

10. The system of claim 9, further comprising means for collecting the patient data of the user.

11. The system of claim 9, wherein the audio or visual signal is transmitted after a predetermined time period has expired.

12. The system of claim 9, wherein the audio or visual signal is transmitted after a predetermined number of attempts to reconnect to the network has failed.

13. A method for notifying a user of moving out of a coverage area, the method comprising the steps of:
providing a central monitoring station in communication with a plurality of telemetry devices in a network;
continuously collecting patient data from the respective telemetry devices to the central monitoring station;
detecting when at least one of the telemetry devices moves out of the coverage area of the network; and,
transmitting an audio or visual signal when the user moves out of the coverage area of the network.

14. The method of claim 13, wherein the detecting step further comprises the step of detecting whether at least one of the telemetry devices loses the connection with the central monitoring station for more than a predetermined time period.

15. The method of claim 14, wherein the audio or visual signal is transmitted after a predetermined number of attempts to reconnect to the network has failed.

16. A device for wirelessly transmitting patient information to a central monitoring station comprising:
a sensory circuit for receiving patient data from a monitoring device;
a transmitter for transmitting the patient data to the central monitoring station, wherein the patient data from the monitoring device, to the sensory circuit, to the transmitter, to the central monitoring station defines a patient data transmission chain; and
a device for producing a signal when the patient data transmission chain is disrupted for a predetermined threshold.

17. The device of claim 16, wherein the predetermined threshold is a predetermined time period.

18. The device of claim 16, wherein the predetermined threshold is a predetermined number of attempts to reestablish the patient data transmission chain.

19. The device of claim 16, wherein the signal is audio, visual, or a combination thereof.

20. The device of claim 16, wherein the signal is transmitted to a separate device.
